SEQUENCE LISTING (4 sequences)

SEQ ID NO: 1 - DNA, 1824 bp, Human
atggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgcagatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccagagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtacaacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatgg

SEQ ID NO: 2 - protein, 607 aa, Human
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTTSSNTISAGWEPNEENEDERDRHLSFSGSGIDDDEDFISSTISTTPRAFDHTKQNQDWTQWNPSHSNPEVLLQTTTRMTADVDRNGTTAYEGNWNPEAHPPLIHHEHHEEEETPHSTSTIQATPSSTTEETATQKEQWFGNRWHEGYRQTPAEDSHSTTGTAAASAHTSHPMQGRTTPSPEDSSWTDFFNPISHPMGRGHQAGRRMDMDSSHSTTLQPTANPNTGLVEDLDRTGPLSMTTQQSNSQSFSTSHEGLEEDLDHPTTSTLTSSNRNDVTGGRRDPNHSEGSTTLLEGYTSHYPHTKESRTFIPVTSAKTGSFGVTAVTVGDSNSNVNRSLSGDQDTFHPSGGHTTHGSESDGHSHGSQEGGANTTSGPIRTPQIPEW

SEQ ID NO: 3 - DNA, 35 bp, Human
tatctagagccgccaccatggacaagttttggtgg

SEQ ID NO: 4 - DNA, 30 bp, Human
tatctagagccattctggaatttggggtgt

(19) United States
(12) Patent Application Publication
(10) Pub. No.: US 2005/0054560 A1
(43) Pub. Date: Mar. 10, 2005

(54) Cd44 variants carrying heparan sulfate chains and uses thereof

(75) Inventors: Avner Yayon, Moshav Sitria (IL); Shlomo Nedvetzki, Jerusalem (IL); David Naor, Jerusalem (IL); Itshak Golan, Ashdod (IL)

Correspondence Address: BROWDY AND NEIMARK, P.L.L.C.;624 NINTH STREET, NW, SUITE 300, WASHINGTON, DC, 20001-5303, US

(73) Assignee: Yissum Research Development Company of the Hebrew University of Jerusalem, Jerusalem (IL)

(21) Appl. No.: 10/486,226

(22) PCT Filed: Aug. 8, 2002
(86) PCT No.: PCT/IL02/00653

Related U.S. Application Data
(60) Provisional application No. 60/310,840, filed on Aug. 9, 2001.

(51) Int. Cl. A61K 38/17; A61K 31/727

(52) U.S. Cl. 514/8; 514/56

(57) ABSTRACT

Modulation of the activity of a heparin-binding growth factor (HBGF) by enhancing or inhibiting high affinity binding of said HBGF to its receptor, can be achieved with an agent selected from: (i) a soluble CD44 isoform carrying at least one chain of a heparan sulfate; (ii) a recombinant chimeric fusion protein comprising the amino acid sequence of a soluble CD44 isoform fused to a tag suitable for proteoglycan purification, said fusion molecule being post-translationally glycosylated to carry at least one chain of a heparan sulfate; and (iii) a sugar molecule being a heparan sulfate derived from a CD44 isoform, or a fragment thereof. The agents (i) and (ii) when the soluble CD44 isoform is the soluble CD44 variant expressed in synovial cells of rheumatoid arthritis patients (CD44vRA), and the heparan sulfate of (iii), are novel.

### FIELD OF THE INVENTION

The present invention relates to the use of proteoglycans and, in particular, of CD44 isoforms bearing heparan sulfate chains, for modulation of the activity of a heparin-binding growth factor, and to some novel CD44 isoforms bearing at least one heparan sulfate chain.

ABBREVIATIONS: AP, alkaline phosphatase; CD44s, standard CD44; CD44v, CD44 variant; ECM, extracellular matrix; FCS, fetal calf serum; FGF, fibroblast growth factor; FGFR, FGF receptor; GAG, glycosaminoglycan; BBGF, heparin-binding growth factor; HS, heparan sulfate; HSPG, heparan sulfate proteoglycan; mAb, monoclonal antibody; OA, osteoarthritis; PG, proteoglycan; RA, rheumatoid arthritis.

### BACKGROUND OF THE INVENTION

The cell surface adhesion glycoprotein, designated CD44, formerly known as lymphocyte homing receptor has been shown to be involved in multiple cellular functions, such as cell-matrix interactions, cell migration, delivery of signals for apoptosis or, conversely, for cell survival and proliferation. In addition, CD44 variants were shown to exert some of their functions through docking and presentation of cytokines, chemokines, enzymes and growth factors to their relevant cell surface receptors or substrates (Naor et al., 1997).

Hyaluronic acid is the principal ligand of CD44, but other cell surface or extracellular matrix (ECM) components, such as osteopontin, fibrinogen, fibronectin, collagen and laminin can interact with this glycoprotein. This multifunctionality of CD44 is possible due to the tremendous structural variability of this receptor derived from its highly complex genetic construction.

CD44 is a single-chain type I transmembrane glycoprotein comprising a conserved extracellular domain (exons 1-5, 15, 16), a nonconserved membrane proximal region, a variable region expressing various combinations of variant exons, a conserved transmembrane segment (exon 17) and a conserved cytoplasmic tail (exon 19). The genomic map of CD44 includes 5 constant exons at the 5′ terminus and 5 constant exons in the 3′ end. In addition to the 10 constant exons, the mouse CD44 gene includes 10 variant exons in the middle of the molecule, designated V₁-V₁₀, resulting in a total of 20 exons. The human CD44 gene comprises only 9 of these 10 variant exons (V₂-V₁₀) thus comprising a total of 19 exons (Screaton et al., 1992). Differential alternative splicing of the variant exons generates many isoforms of CD44 that express various combinations of variant exons (designated Exon Vx, x=1-10), which are inserted in the membrane proximal extracellular domain and constitute the variable region of the molecule. These CD44 variant isoforms (CD44v) are designated by the variant exons that they include, e.g. CD44v3, CD44v6, CD44v8, CD44v8,9, CD44v9, CD44v10, CD44v3-v10, etc.

Theoretically, hundreds of CD44 isoforms can be generated by alternative splicing of the 10 (mouse) or the 9 (human) variant exons inserted in different combinations between the two constant exon regions, 5 exons in each end of the molecule. However, the number of the known CD44 variants (CD44v) has so far been limited to a few dozen, detected mostly on epithelial cells, keratinocytes, activated leukocytes and many types of tumor cells.

Differential utilization of the 10 (mouse) or the 9 (human) variant exons as well as variations in N-glycosylation, O-glycosylation, and glycosaminoglycanation (by heparan sulfate or chondroitin sulfate), generate multiple isoforms of different molecular sizes (85-230 kDa). The smallest CD44 molecule (85-95 kDa), which results from direct splicing of constant exon 5 to constant exon 16 and thus lacks the entire variant region, is standard CD44 (CD44s), expressed mainly by hematopoietic cells and is also known as hematopoietic CD44 (CD44H). Soluble isoforms of CD44 (sCD44), the shed ectodomain of transmembrane CD44, lack the transmembrane and the cytoplasmatic tail of CD44 (Stickeler et al., 2001). The soluble form of CD44H has a molecular weight of 70-80 kDa. Although serum concentrations of soluble CD44 vary widely in healthy individuals, elevated levels of soluble CD44 have been identified in synovial fluid from rheumatoid arthritis patients and in patients with non-Hodgkin's lymphoma.

The involvement of CD44 protein and variants thereof in autoimmune diseases is known, and several anti-CD44 monoclonal antibodies (mAbs) directed against the constant (anti-pan CD44 mAbs) or other regions of CD44 have been suggested as agents for treatment of various autoimmune diseases, particularly diseases of the rheumatic type (EP 538754, EP 501233, WO 9500658, WO 9409811).

Marked accumulation of CD44, and sometimes hyaluronic acid, is detected in areas of intensive cell migration and cell proliferation as in wound healing, tissue remodeling, inflammation (including autoinflammation), morphogenesis and carcinogenesis. The involvement of CD44 in malignant processes has also been described by the present inventors (Naor et al., 1997). Anti-CD44 mAbs injected into mice were shown to inhibit or prevent infiltration of various lymphoma and carcinoma cells into their target organs.

It has been reported that mAbs directed against the constant epitopes shared by all CD44 isoforms (anti-pan CD44 mAbs), induced resistance to several experimental inflammatory autoimmune diseases, such as collagen-induced arthritis (Nedvetzki et al., 1999), experimental allergic encephalomyelitis (EAE) (Brocke et al., 1999) and insulin-dependent diabetes mellitus (IDDM) (Weiss et al., 2000). However, targeting of CD44 constant epitopes with anti-pan CD44 mAbs may also limit the CD44-dependent physiological commitment of normal cells expressing such epitopes. In contrast, targeting of CD44 alternatively spliced variant epitopes or products of CD44 sequence alterations generated by “inaccurate” alternative splicing (as found in some cancer cells and in synovial fluid cells of RA patients) with specific mAbs could restrictively block the activity of pathological cells, i.e. the inflammatory cells found in autoimmune diseases or of cancer cells. This is conceivable because normal cells may express CD44s, CD44 isoforms expressing different variant exons or CD44 lacking the sequence alteration.

Proteoglycans (PGs) are large and complex macromolecules comprised of numerous molecules of a core protein and long chains of modified sugars called glycosaminoglycans (GAGs). More specifically, GAGs are large complexes of polysaccharide chains associated with a core protein in which the polysaccharide makes up most of the mass, often 95% or more. These compounds have the ability to bind large amounts of water, thereby producing a gel-like matrix that forms the body's ground substance. GAGs stabilize and support cellular and fibrous components of tissue while maintaining the water and salt balance of the body. The combination of insoluble protein and the ground substance forms connective tissue. For example, cartilage is rich in ground substance while tendon is composed primarily of fibers.

GAGs are long chains composed of repeating disaccharide units of monosaccharides (aminosugar-acidic sugar repeating units). The aminosugar, typically N-acetylglucosamine or N-acetylgalactosamine, may also be sulfated. The acidic sugar may be D-glucuronic acid or L-iduronic acid. GAGs, with the exception of hyaluronic acid, are covalently bound to a protein, forming proteoglycan monomers. The covalent attachments between GAGs and a core protein are glycosidic bonds between sugar residues and the hydroxyl groups of Ser residues in the protein.

The carbohydrate structure of GAGs varies markedly among different tissues and proteoglycans, with differing patterns of sulfation, carboxyl groups, and N-acetylation on uronic acid or other carbohydrate structures. All GAGs contain hexosamine or uronic acid derivative products of the glucose pathway and from exogenous glucosamine, for example: hyaluronic acid (HA) contains N-acetylglucosamine+glucuronic acid; keratan sulfate contains sulfated N-acetylglucosamine+galactose; chondroitin sulfate (CS) contains glucuronic acid+sulfated N-acetylgalactosamine; heparan sulfate (HS) contains sulfated glucosamine+glucuronic or iduronic acid; dermatan sulfate contains sulfated iduronic acid+galactosamine.

Heparin and heparan sulfate consist of alternate sequences of an uronic acid (iduronic or glucuronic) and N-acetylglucosamine, variously sulfated depending on the tissue and the animal species from which they have been obtained and, to a certain extent, on the isolation processes too. Heparan sulfate GAGs are found in many tissues—some are located in connective tissue and basal lamina, while others are moieties of surface proteins that are either integral to membranes, or extracellular, anchored to the cell by a glycosylphosphatidylinositol (GPI) linkage. Heparan sulfates, as components of proteoglycans, probably play important roles in cell-cell interactions.

A number of growth factors including members of the fibroblast growth factor (FGF), colony-stimulating factor (CSF), transforming growth factor beta (TGF-β), interleukin (IL), and bone morphogenetic protein (BMP) families, heparin-binding epidermal-like growth factor (HB-EGF), insulin-like growth factor, vascular endothelial growth factor (VEGF), macrophage inflammatory protein-1β (MIP-1β), regulated on activation, normally T cell expressed and secreted (RANTES) and hepatocyte growth factor (HGF), have been shown to bind to ECM and HS. For example. FGFs bind avidly to heparin and to heparan sulfate proteoglycans (HSPGs) found on cells and in the ECM. Studies on the mode of action of FGF-2 identified a novel role for heparin-like molecules in the formation of distinct FGF-2-heparin complexes that are essential for binding of FGF-2 to its cognate receptor (Yayon et al., 1991) and subsequent signal transduction (Rapraeger et al., 1991). The crucial role of the cell surface HS was revealed by the finding that high affinity receptor binding of FGF-2 was abolished in Chinese hamster ovary (CHO) mutant cell lines defective in their metabolism of glycosaminoglyans, and that receptor binding was restored upon addition of exogenous heparin (Yayon et al., 1991).

In the case of CD44, while alternative splicing is a most efficient machinery for the enrichment of the genetic information stored in a single gene, post-translation modifications by glycosylation and GAG attachments further modifies the CD44 protein, thus allowing further expansion of its functions. To this end, it was found that HS attached to the v3 exon of v3-containing CD44 PGs can bind HS-binding chemokines and growth factors. The binding of HS-binding growth factors to the CD44 proteoglycan allows frequent attachments between low affinity, high density HS-ligand complexes and their unoccupied, less abundant high affinity receptors expressed on the same cell, or more oriented and efficient presentation of the growth factor to the relevant receptor expressed on a different cell, resulting in input of transduced signals and output of cell activity (e.g., cell proliferation).

The growth factor binding function of v3-containing CD44 can support both physiological (e.g. embryonic limb outgrowth) and pathological (e.g. tumor cell motility and growth) activities. As mentioned above, we and others have shown that CD44 targeting by anti-CD44 mAbs can reduce experimental tumor growth as well as pathological activities in experimental autoimmune diseases, possibly by interfering with CD44-dependent growth factor presentation, as well as disruption of other CD44-dependent functions (for example, cell migration). However, in most cases, the mAbs were directed against standard CD44 epitopes, shared by all CD44 isoforms, resulting in targeting of cells engaged in both physiological and pathological activities. On the other hand, if cells engaged in pathological activities express CD44 isoforms that are not expressed on normal cells, mAbs exclusively recognizing the CD44 variants associated with the pathological activities may reduce the disease activity with minimal damage to innocent normal cells.

To test the hypothesis that disease-specific CD44 is expressed on cells involved in pathogenesis, the present inventors have previously analyzed the CD44 repertoire of synovial cells from RA patients by flow cytometry and the reverse transcriptase-polymerase chain reaction (RT-PCR). The CD44 RT-PCR products were isolated and sequenced to define the pathological CD44 mRNA. As disclosed in PCT Publication WO 00/75312, herein incorporated by reference as if fully disclosed herein, the mRNA of synovial cells from inflamed joints includes a dominant isoform, CD44v3-v10, which is also present in normal keratinocytes. The CD44v3-v10 transcript was detected in 44 of 47 patients. When the CD44v3-v10 isoform of RA synoviocytes was sequenced and its sequence was compared with the published sequence of CD44v3-v10 (Screaton et al., 1992 and 1993), it was found that it included an extra trinucleotide sequence (CAG), that was illegitimately transcribed from the end of the intron bridging exon v4 to exon v5 and inserted at the 5′ end of exon v5, allowing it to encode the hydrophobic amino acid alanine, without interfering with the entire reading frame. The translation at both sides of the new insert is not changed, as the original GAT codon (which encodes aspartic acid) is preserved as are all the other codons of exons v4 and v5. The location of the extra CAG in the CD44 transcript of the RA patient's synoviocytes is shown below:

| | |
|---|---|
| Normal CD44: | |
| Exon V4 . . . GGATGACTG | |
| ATGTAGACA . . . Exon V5 | |
| RA CD44: | |
| Exon V4 . . . GGATGACTG CA | |
| GATGTAGACA . . . Exon V5 | |
| | Ala |

A transcript with identical CAG insertion was detected in synovial cells of 20 out of 26 RA patients who displayed the CD44v3-v10 transcript. This CD44v of the RA patients was, therefore, designated CD44vRA. This CD44 variant is a naturally occurring molecule which has not been detected in cells of healthy individuals but only in those of RA patients.

As disclosed in the above-mentioned WO 00/75312, the expressed CD44vRA enables production of CD44vRA-specific mAbs, that can be used for prevention and treatment of infectious and other inflammatory diseases, cancer and autoimmune diseases, particularly rheumatoid arthritis.

### SUMMARY OF THE INVENTION

The present invention relates, in one aspect, to a pharmaceutical composition for modulation of the activity of a heparin-binding growth factor (HBGF) by enhancing or inhibiting high affinity binding of said HBGF to its receptor, comprising a pharmaceutically acceptable carrier and an agent selected from:
(i) a soluble CD44 isoform carrying at least one chain of a heparan sulfate;
(ii) a recombinant chimeric fusion protein comprising the amino acid sequence of a soluble CD44 isoform fused to a tag suitable for proteoglycan purification, said fusion molecule being post-translationally glycosylated to carry at least one chain of a heparan sulfate; and
(iii) a sugar molecule consisting of a heparan sulfate derived from a CD44 isoform or a fragment thereof.

In another aspect, the present invention relates to the use of an agent (i), (ii) or (iii) as defined above for the preparation of a pharmaceutical composition for modulation of the activity of a heparin-binding growth factor (HBGF) by enhancing or inhibiting high affinity binding of said HBGF to its receptor.

In a further aspect, the invention relates to novel agents (i) and (ii) as defined above wherein the soluble CD44 isoform is the soluble CD44vRA, and to the novel sugar molecules as defined in (iii) above wherein the heparan sulfate is derived from any CD44 isoform, preferably from the CD44vRA, or a fragment thereof, said heparan sulfate and fragments being capable of modulating the activity of a heparin-binding growth factor (HBGF).

In one embodiment, the HBGF is a member of the FGF family, for example, FGF-2, and the agent of the invention can either enhance or inhibit FGF receptor binding, depending on the structure of said HS.

In the proteoglycan CD44vRA, as in all heparan sulfate proteoglycans (HSPGs), there are several linear HS chains covalently attached to a protein core. According to one embodiment, the invention relates to a sugar molecule being a heparan sulfate derived from CD44vRA, that may have for example at least one highly sulfated domain. In one embodiment, said at least one HS chain isolated from the proteoglycan CD44vRA is not associated with the core protein of said CD44vRA. In another embodiment, the said at least one HS chain may be associated with the core protein of said CD44vRA.

In another embodiment, the invention relates to a heparan sulfate as defined above or a fragment thereof, preferably containing at least 2, more preferably at least 5 or 6, most preferably 10-16, monosaccharide residues.

In a further aspect, the invention further relates to an inhibitor of a heparan sulfate or a fragment thereof as defined above such as an antibody, a peptide or an oligosaccharide or polysaccharide mimetic. These inhibitors will, for example, inhibit angiogenesis thus being useful for inhibition of cell proliferation and migration in the treatment of primary tumors and metastasis, or in treatment of destructive inflammatory disorders.

### BRIEF DESCRIPTION OF THE FIGURES

FIG. 1 shows the insertion of the trinucleotide CAG in the CD44v3-v10 sequence of RA patients. CD44v3-v10, cloned from RNA of RA synovial fluid cells (CD44vRA) was subjected to nucleotide sequencing. Alignment of CD44vRA with normal CD44v3-v10 (Screaton et al.) revealed extra CAG, transcribed from the end of the intron bridging variant exon v4 to variant exon v5, precisely at the splicing junction. The figure shows the position of CAG insertion, which allows translation of alanine without interfering with the entire reading frame. Identical sequence modification was detected in 20 of 26 RA patients.

FIGS. 2A-2B show binding of anti-CD44 mAbs to CD44-Namalwa transfectants. FIG. 2A are graphs showing the ability of the Namalwa transfectants (Namalwa Neo, Namalwa CD44s, Namalwa CD44v3-v10, and Namalwa CD44vRA) to interact with anti-pan-CD44 mAb and anti-CD44v6 mAb as analyzed by flow cytometry, using fluorescein-labeled anti-mouse Ig to detect the binding of the antibodies to cell surface CD44 epitopes. FIG. 2B depicts Western blot analysis of cell extracts from Namalwa transfectants with Hermes-3 mAb that confirms the flow cytometry findings. The anti-CD44 mAb detected almost equal expression of CD44. variant exon product on Namalwa-CD44v3-v10 and Namalwa-CD44vRA, and standard CD44 exon product on Namalwa-CD44s.

FIGS. 3A-3B show that Namalwa-CD44v3-v10 and Namalwa-CD44vRA similarly bind FGF-2. FIG. 3A. The Namalwa transfectants Namalwa Neo, Namalwa CD44s, Namalwa CD44v3-v10, and Namalwa CD44vRA -were incubated with biotinilated FGF-2 and then analyzed by flow cytometry for their ability to bind FGF-2, as detected by staining with streptavidin-PE. Control: Namalwa cells transfected with empty vector (Namalwa-Neo) and incubated with biotinilated FGF-2 antibody. FIG. 3B. Western blot analysis of cell extracts from Namalwa transfectants with anti-FGF-2 antibody confirmed the flow cytometry analysis. The Namalwa transfectants were preincubated with FGF-2 before subjection to cell extraction and gel electrophoresis. The anti-FGF-2 antibody detected similar binding of FGF-2 to CD44v3-v10 and CD44vRA, whereas CD44s did not bind FGF-2.

FIGS. 4A-4H show that FGF-2 is bound to CD44 proteoglycan via heparan sulfate. FIGS. 4A-4D. Excess of soluble heparin blocks the binding of FGF-2 (bFGF) to Namalwa-CD44vRA. Namalwa-CD44vRA cells were coincubated with biotinilated FGF-2 and excess of soluble heparin or soluble chondroitin sulfate A+C, and then analyzed by flow cytometry for their ability to bind FGF-2. The binding of biotinilated FGF-2 was detected with streptavidin-PE. The first histogram depicts Namalwa-CD44vRA cells incubated with streptavidin-PE only. FIGS. 4B-4D show similar results observed with three individual clones of Namalwa-CD44vRA cells: 10vRA, 15vRA and 20vRA, respectively. FIG. 4E. Treatment with the degrading enzyme heparinase reduced FGF-2 binding to Namalwa-CD44vRA. Namalwa-CD44vRA cells were treated with heparinase or chondroitinase ABC, and then analyzed by flow cytometry for their ability to bind biotinilated FGF-2. Detection system and control were as in FIG. 4A. FIGS. 4F-4H show similar results observed with three individual clones of Namalwa-CD44vRA cells: 10vRA, 15vRA and 20vRA, respectively.

FIGS. 5A-5B show enhanced binding of FGF receptor 1 to Namalwa-CD44vRA. FIG. 5A shows binding of FGF receptor 1 to Namalwa transfectants. Namalwa-Neo (Nam-Neo), Namalwa-CD44v3-v10 (Nam-v3-10) and Namalwa-CD44vRA cells (Nam-vRA) were incubated in the presence of FGF-2 with soluble FGF receptor 1 conjugated to alkaline phosphatase. The ability of the receptor to bind to FGF-2 presented by Namalwa transfectants was detected with alkaline phosphatase substrate at O.D. 405. FIG. 5B shows binding of FGF-2 to Namalwa transfectants. FGF-2 conjugated to alkaline phosphatase was used to analyze the direct binding of FGF-2 to the same Namalwa transfectants as in FIG. 5A under identical experimental conditions. A representative experiment of three experiments.

FIG. 6 shows that FGF-2 bound to Namalwa-CD44vRA induces enhanced proliferation in Baf-32 cells expressing FGF receptor 1. The indicated fixed Namalwa transfectants were incubated in the presence of FGF-2 with Baf-32 cells. The ability of the bound FGF-2 to induce proliferation in Baf-32 cells was analyzed by MTS at O.D. 490. Positive control: incubation of Baf-32 cells with FGF-2 and heparin. Negative controls: incubation of Baf-32 cells with FGF-2 alone or with heparin alone. Inset: A similar experiment, except that the proliferation of the positive control Baf-32 cells was adjusted to the proliferation level of Baf-32 cells incubated with Namalwa-CD44vRA cells. A representative experiments of total 4 experiments.

FIGS. 7A-7D show that synovial fluid cells from RA patients bind soluble FGF receptor-1 via CD44 receptor. FIG. 7A. CD44 expression on synovial fluid cells from an RA patient and joint cells from an osteoarthritis (OA) patient. Cells collected from joints of an RA or an OA patient were analyzed by flow cytometry with anti-pan CD44 mAb, anti-CD44v3 mAb or anti-CD44v6 mAb. First histogram in each panel indicates staining with second antibody alone. Similar flow cytometric histograms were recorded in 11 RA patients and 6 OA patients. FIG. 7B. Enhanced binding of soluble FGF receptor-1 to synovial fluid cells of RA patients. Cells from the joints of RA (11 samples) and OA (6 samples) patients were incubated with soluble FGF receptor-1 conjugated to alkaline phosphatase in the presence (not shown) or the absence (FIG. 7B) of FGF-2. The interaction of the FGF receptor-1 with the FGF-2, bound to the joint cells, was analyzed at O.D. 405 by detection system that includes the alkaline phosphatase substrate. Similar results were observed in the presence of FGF-2. Insets: Binding of anti-FGF antibody to joint cells of RA (a) and OA (b) patients to evaluate the FGF inclusion in these cells. First histogram in each inset indicates staining with second antibody only (goat anti-rabbit Fab′-FITC). The equal inclusion of FGF in RA and OA cells was confirmed in an additional 11 RA samples and 6 OA samples. FIG. 7C. The binding of soluble FGF receptor-1 to synovial fluid cells of RA patients is CD44v3-associated. Synovial fluid cells from three RA patients (RA6, RA8, RA10) were incubated with soluble FGF receptor-1 conjugated to alkaline phosphatase in the presence of medium (1), isotype-matched control immunoglobulin (2), or 1 μg (3), 300 ng (4), 30 ng (5) and 1 ng (6) anti-CD44v3 mAb. The interaction of the FGF receptor-1 with FGF-2, bound to the joint cells, was analyzed as indicated in FIG. 7B. Anti-CD44v3 mAb inhibits the binding of soluble FGF receptor-1 to the synovial fluid cells in a dose-dependent manner. The highest concentration (1 μg) of anti-CD44v3 mAb inhibited FGF receptor-1 binding to synovial fluid cells from additional samples of 4 RA patients. FIG. 7D. Anti-CD44v3 mAb, but not anti-CD44mAb directed against a constant epitope, inhibited the binding of soluble FGF-receptor 1 to synovial fluid cells of RA patients. Synovial fluid cells from three RA patients (RA1, RA3, RA6) were incubated with soluble FGF-receptor 1 conjugated to alkaline phosphatase in the presence of medium (1), isotype matched control immunoglobulin (2), 1 μg anti-CD44v3 mAb (3), and 1 μg anti-pan CD44mAb (4). The interaction of the FGF receptor -1 with FGF-2 bound to the joint cells was analyzed as described in FIG. 7B.

FIG. 8 depicts schematically the construct of the pCX-Fc-CD44 zeovectors used for expression of the soluble fusion proteins comprising the sequence of the soluble CD44s, CDv3-v10 or CD44vRA fused to the Fc region of the gamma globulin heavy chain. The CD44 cDNAs (RT-PCR products) were cloned into the pCXFc zeovector in the NheI site. The vector digested with NheI restriction enzyme and the RT-PCR products were digested with XbaI restriction enzyme. The restricted vector and the cDNA were ligated with T4 ligase (Promega).

FIG. 9 depicts Western blot analysis of supernatants from 293T cells transfected with the constructs of FIG. 8, with the anti-CD44 Hermes-3 mAb (S-CD44s, R—CD44vRA, V —CD44v3-v10, C—no transfection).

### DETAILED DESCRIPTION OF THE INVENTION

While alternative splicing is a most efficient machinery for the enrichment of the genetic information stored in a single gene, post-translation modifications by glycosylation and GAG attachments further modifies the CD44 protein, thus allowing further expansion of its functions. It has been reported that CD44 isoforms containing the variant exon v3 product are decorated with heparan sulfate (HS) and are thus capable of binding a heparin-binding growth factor (HBGF) such as FGF-2 (Naor et al., 1997). It can be conceived that the binding of the HBGF is done in a way that the HS can present it and induce autocrine or paracrine activities of the corresponding receptors. Therefore, blocking, for example, CD44 v3-specific HS-FGF-FGFR interaction, may reduce the activity of HS-recognizing growth factors which mediate pathological functions, e.g. destructive joint inflammation in RA patients.

The binding of HS-binding growth factors to the CD44 proteoglycan allows frequent attachments between low affinity, high density HS-ligand complexes and their unoccupied, less abundant high affinity receptors expressed on the same cell, or more oriented and efficient presentation of the growth factor to the relevant receptor expressed on a different cell, resulting in input of transduced signals and output of cell activity (e.g., cell proliferation).

The present invention relates, in one aspect, to a pharmaceutical composition for modulation of the activity of a heparin-binding growth factor (HBGF) by enhancing or inhibiting high affinity binding of said HBGF to its receptor, comprising a pharmaceutically acceptable carrier and an agent selected from:
(iv) a soluble CD44 isoform carrying at least one chain of a heparan sulfate;
(v) a recombinant chimeric fusion protein comprising the amino acid sequence of a soluble CD44 isoform fused to a tag suitable for proteoglycan purification, said fusion molecule being post-translationally glycosylated to carry at least one chain of a heparan sulfate; and
(vi) a sugar molecule consisting of a heparan sulfate derived from a CD44 isoform, or a fragment thereof.

In another aspect, the present invention relates to the use of an agent (i), (ii) or (iii) as defined above for the preparation of a pharmaceutical composition for modulation of the activity of a heparin-binding growth factor (IBGF) by enhancing or inhibiting high affinity binding of said HBGF to its receptor.

The HBGF may be any heparin-binding growth factor such as, but not limited to, a growth factor selected from a member of the fibroblast growth factor (FGF) family, e.g. FGF-2 or any of the FGF-1 to FGF-22 factors; a member of the colony-stimulating factor (CSF) family, e.g. CSF-1, G-CSF, M-CSF, GM-CSF; a member of the transforming growth factor beta (TGF-β) family; a member of the interleukin (IL) family, e.g. IL-1 or any of the IL-1 to IL-27 molecules; a member of the bone morphogenetic protein (BMP) family; heparin-binding epidermal-like growth factor (HB-EGF); insulin-like growth factor (IGF); vascular endothelial growth factor (VEGF); macrophage inflammatory protein-1β (MIP-1β); regulated on activation, normally T cell expressed and secreted (RANTES); and hepatocyte growth factor (HGF).

In one embodiment, the pharmaceutical composition of the invention is intended for modulating heparin-dependent growth factor activity relevant for promoting tissue-specific cell proliferation, migration and differentiation. According to this embodiment, the composition may be used for induction of angiogenesis and blood vessel formation, bone fracture healing, enhancement of wound healing, treatment of ischemic heart diseases and peripheral vascular diseases, neuronal regeneration, and promotion of tissue regeneration, for example liver regeneration, or promotion of tissue regeneration after transplantation of myocytes into heart tissues or of dopaminergic/neuronal cells into brain tissue.

In another embodiment, for the purposes mentioned above, the composition may be administered in combination with a HBGF selected from a FGF, a CSF, a TGF-β, an IL, VEGF, MIP-1β, BMP, IGF, HB-EGF, RANTES and HGF. The HBGF may be administered together, before or after the agent of the invention. For example, the agent may be administered with FGF-2 for treatment of heart failure by transplantation of myocytes into heart tissues or for tissue regeneration after transplantation of dopaminergic/neuronal cells into brain tissue; with FGF-2 and/or VEGF for induction of angiogenesis, treatment of ischemic heart disease or peripheral vascular disease; with HGF for promoting liver regeneration; or with FGF-7 (known formerly as keratinocyte growth factor) for enhancement of wound healing.

In a further embodiment, the composition of the invention can be used for prevention and treatment of infectious and other inflammatory diseases; autoimmune diseases such as multiple sclerosis, Chron's disease, ulcerative cholitis, insulin-dependent diabetes mellitus (IDDM) and, preferably, rheumatoid arthritis; and a CD44-dependent cancer such as non-Hodgkin's lymphoma, a melanoma or colon-rectal cancer.

Any soluble CD44 isoform is encompassed by the invention provided that it contains at least one heparan sulfate chain. Examples are the soluble CD44s, any of the soluble CD44 variants, preferably a CD44v including the exon 3 such as CD44v3, more preferably the CD44v3-v10 isoform, and most preferably, the soluble CD44vRA, encoded by the nucleotide sequence of SEQU ID NO: 1 and having the amino acid sequence of SEQU ID NO:2 herein. The soluble CD44vRA is herein disclosed for the first time.

Thus, in one preferred embodiment of the present invention, the pharmaceutical composition is used for the treatment of rheumatoid arthritis and comprises CD44vRA carrying at least one chain of a heparan sulfate.

The soluble CD44 isoforms and CD44 fusion proteins for use in the present invention can be prepared by well known techniques, for example as described in WO 01/40267.

The soluble CD44 isoforms do not contain the transmembrane and cytoplasmic domains of CD44, and are encoded by the nucleotide sequence 1-1824 from the published sequence of CD44 (Screaton et al., 1992) with the corresponding variations according to the presence or absence of one or more of the variant exons, and the soluble CD44vRA is encoded by the nucleotide sequence of SEQU ID NO:1 herein.

Thus, CD44 coding sequences may be obtained by RT-PCR cloning by standard methods well-known in the art. If necessary, the desired CD44 domain can then be excised by restriction enzyme digest or by PCR using appropriate oligonucleotide primers. The so obtained sequences may then be fused to a suitable tag to form the DNA sequences coding for the desired recombinant CD44 fusion protein. Any tag suitable for proteoglycan purification may be used according to the invention including, but not being limited to, glutathione S-transferase (GST), polyHis, and, more preferably, the Fc region of the human globulin heavy chain, e.g. human IgG1.

The post-translational glycosylation occurs when the cloned DNA is expressed in suitable mammalian cells including, but not limited to, endothelial, fibroblast and epithelial cells, such as embryonic kidney cells, ovary cells, e.g. Chinese hamster ovary cells (CHO), or aortic endothelial cells. For the purpose of experimental studies, transient transfection can be performed into COS cells or into 293T cells, a derivative of the human renal 293 epithelial cell line which is transformed by adenovirus E1A gene product and which also expresses SV40 large T antigen.

When expressed as a fusion protein, the ectodomain of the CD44 molecule will usually be cleaved from the fusion partner.

In one preferred embodiment of the invention, the composition comprises a recombinant chimeric fusion protein wherein soluble CD44vRA (SEQU ID NO:2) is fused to the Fc region of the gamma globulin heavy chain (CD44vRA-Fc).

The CD44 isoforms according to the invention are defined as carrying at least one heparan sulfate chain, but they may carry more HS chains, for example 2, 3 or more than 3 chains. In a preferred embodiment, the at least one heparan sulfate chain has at least one highly sulfated domain. The HS chain may contain at least 2, preferably at least 5 or 6, and up to 10-16, monosaccharide residues.

In a further embodiment, the composition of the invention comprises a sugar molecule being a heparan sulfate derived from a CD44 isoform such as CD44s, CD44v3-v10, or, preferably, CD44vRA, or a fragment of said sugar. In a preferred embodiment, the sugar heparan sulfate chain has at least one highly sulfated domain. The HS chain or fragment may contain at least 2, preferably at least 5 or 6, and up to 10-16, monosaccharide residues.

The heparan sulfate of the invention can be prepared by standard processes such as by controlled chemical treatment or by protease treatment of the proteoglycan CD44 isoform, for example as described in Nader et al., 1987. Examples of methods used for preparation of heparan sulfates and their characterization are described in Aviezer et al., 1994. Once obtained by chemical or proteolytic treatment, the mixtures of HS can be resolved into their individual components by many of the techniques useful also in protein and amino acid separation: differential centrifugation, ion-exchange chromatography and gel filtration. For characterization of the chemical composition, the carbohydrate molecule can be subjected to hydrolysis in strong acid yielding a mixture of monosaccharides, which, after conversion to suitable volatile derivatives, may be separated, identified and quantified by gas-liquid chromatography to yield the overall composition of the polysaccharide.

The HS fragments according to the present invention can be prepared from the HS preparations either by chemical or by enzymatic degradation according to methods well-known in the art as, for example, the methods described for degradation of heparin in Aviezer et al., 1994 and in U.S. Pat. No. 6,020,323, both documents being herein incorporated by reference as if fully described herein. Thus, the HS fragments can be produced in several different ways: controlled chemical (by nitrous acid or peliodate oxidation) or enzymatic (by heparinases, heparanases, or heparitinases) depolymerization. The conditions for depolymerization can be carefully controlled to yield fractions or fragments of desired molecular weights. Nitrous acid depolymerization is commonly used.

For the enzymatic degradation, several types of enzymes can be used such as heparitinase I or II (commercially available from Seikagaku Co., Tokyo, JP), or heparanase enzymes such as MM5, a mammalian heparanase from human placentas (commercially available from Rad-Chemicals, Weizmann Industrial Park, Ness Ziona, Israel), or PC3, a bacterial endoglycosidase.

The HS fragments can then be separated, purified and characterized by standard methods used in carbohydrate chemistry. For example, a carbazole assay performed in a manner similar to that disclosed by Carney, S. L. in Proteoglycan Analysis, A Practical Approach, Chaplin, M. F. and Kennedy, J. F. (Eds.) IRL Press, Oxford, Washington, D.C. (1986) p. 129, can be utilized to determine the amount of oligosaccharide material present (e.g., amount of sugar present) in a given test sample. Picogram (pg) quantities of sugar can be quantified in this manner.

For screening of the sugar fragments that have the desired biological activity of binding and presentation of the HBGF to its receptor, one of the biological assays described in Aviezer et al., 1994, can be used. In one embodiment, the assay is carried out using either the immobilized soluble receptor or the immobilized ligand. Soluble receptors can be produced as receptor-tag fusion proteins, wherein said tag may be alkaline phosphatase (AP), e.g. FGFR1-AP. Immobilization can be achieved, for example, by biotinilation and binding to avidin or streptavidin. In another embodiment, the assay is carried out using heparan sulfate-deficient cells such as Namalwa cells or a CHO mutant cell line such as CHO pgs A745.

According to the present invention, CD44v3-v10 and CD44vRA, expressed either as an integral transmembrane proteoglycan or in a soluble secreted form, efficiently enhanced high affinity binding of FGF-2 to its receptor FGFR1. These results indicate that these CD44 isoforms carry HS chains and play an important role in modulating FGF-FGFR binding and signaling in vivo. The effect of these CD44 isoforms is dependent on the HS chains and, therefore, elimination of the HS chains by treatment with heparinase, completely abolished the effect (but not by treatment with chondroitinase).

In another aspect, the present invention comprises an agent capable of modulating the activity of a heparin-binding growth factor (HBGCF) by enhancing or inhibiting high affinity binding of said HBGF to its receptor, said agent being selected from:
(i) the soluble CD44vRA variant expressed in synovial cells of rheumatoid arthritis (RA) patients (herein soluble CD44vRA of SEQ ID NO:2), carrying at least one chain of a heparan sulfate;
(ii) a recombinant chimeric fusion protein comprising the SEQ ID NO:2 fused to a tag suitable for proteoglycan purification, said fusion molecule being post-translationally glycosylated to carry at least one chain of a heparan sulfate; and
(iii) a sugar molecule being a heparan sulfate derived from a CD44 isoform, or a fragment thereof.

As defined above with regard to the ingredients of the compositions of the invention, the agent (ii) of the invention may be a recombinant chimeric fusion protein wherein the amino acid sequence of the soluble CD44vRA (SEQ ID NO:2) is fused to a tag selected from the Fc region of the gamma globulin heavy chain, glutathione S-transferase (GST) or polyHis, and is preferably soluble CD44vRA fused to the Fc region of the gamma globulin heavy chain (CD44vRA-Fc), and the agent (iii) may be a HS molecule or a fragment thereof having at least 2, 5 or 6, and up to 10-16 monosaccharide residues and has preferably at least one highly sulfated domain.

In still another aspect, the present invention relates to an inhibitor of a sugar HS molecule as defined above, wherein said inhibitor is a molecule which inhibits the biological activity attributed to the HS molecule, either alone or coupled to the CD44 isoform. The inhibitor may be an antibody, a peptide, an oligosaccharide or a polysaccharide mimetic, and may be screened by well known methods for screening inhibitors or antagonists, for example by using available phage display libraries.

Methods for prevention or treatment of diseases or disorders that are associated to an heparin-binding growth factor (HBGF) binding to its receptor and that can be prevented or treated by enhancing or inhibiting high affinity binding of a HBGF to its receptor, are also encompassed by the present invention by administration to an individual in need thereof of an effective amount of an agent selected from:
(i) a soluble CD44 isoform carrying at least one chain of a heparan sulfate;
(ii) a recombinant chimeric fusion protein comprising the amino acid sequence of a soluble CD44 isoform fused to a tag suitable for proteoglycan purification, said fusion molecule being post-translationally glycosylated to carry at least one chain of a heparan sulfate; and
(iii) a sugar molecule being a heparan sulfate derived from a CD44 isoform, or a fragment thereof.

In yet a further aspect, the present invention provides a method for diagnosis of rheumatoid arthritis in an individual comprising:
(i) obtaining a sample from the joints of the individual;
(ii) contacting said sample with a FGFR conjugated to a detection system;
(iii) detecting the presence of CD44vRA expressed by the joint cells in the sample through binding of an endogenous or exogenous FGF and the conjugated receptor by adding a substrate for the detection system, whereby development of color indicates the presence of CD44vRA expressed by the joint cells and a high probability of rheumatoid arthritis.

The sample used for the method is preferably synovial fluid containig synovial cells that express CD44vRA. The detection system may be, for example, alkaline phosphatase (AP).

The invention further provides a diagnostic kit for RA for identifying the disease or for follow up of treatment, said kit comprising the suitable FGFR, for example FGFR-1, conjugated to a detection system, for example, AP (FGFR1-AP), a substrate for the detection system, for example, p-nitrophenyl phosphate (NPP) for AP, and directions for its use.

The invention will now be illustrated by the following non-limiting examples.

### EXAMPLES

### Materials and Methods

(i) Preparation of hCD44v3-10 and hCD44vRA Plasmids

The entire human hCD44v3-10 cDNA was cloned from human keratinocyte total RNA by reverse transcriptase-polymerase chain reaction (RT-PCR) amplification, using the two primers assigned from the published CD44 sequence, including the Xbal recognition site. The primers are described in WO 00/75312. The PCR product was digested with Xbal enzyme and ligated into a pcDNA3.1 Neovector (Invitrogen).

Using the same protocol the entire hCD44vRA cDNA was cloned from cellular total RNA extracted from synovial fluid cells of rheumatoid arthritis patients and CD44s was cloned from Hela cells. The pcDNA3.1 neovector alone served as control.

ii) Stable Transfection of the Plasmids Into Namalwa Cells

Namalwa cells (derived from patients with Burkitt's lymphoma), lacking CD44, were transfected with: (1) standard CD44 cDNA isolated from Hela cells (designated Namalwa CD44s); (2) CD44v3-v10 cDNA isolated from keratinocytes (Namalwa-CD44v3-v10 or Namalwa-CD44v); (3) CD44v3-v10 cDNA containing the extra CAG isolated from RA synoviocytes (Namalwa-CD44vRA); or (4) with empty vector (Namalwa-pcDNA 3.1 or Namalwa-NEO).

For this purpose, CD44s, CD44v3-v10 and CD44vRA cDNAs were cloned in an expression vector and transfected into Namalwa that do not express CD44. Thus, a mixture of 30 μg polybrene and 10 μg of plasmid pcDNA3.1, standard CD44, CD44v3-10 or CD44vRA cDNAs were added to 5×10⁵ Namalwa cells (CD44 negative cells) suspended in 3 ml RPMI 1640 medium. After 12-24 h incubation, the cells were treated with 30% DMSO for 3 min, washed and incubated in RPMI 1640 medium containing 10% FCS for 48 h. Stable transfectants were generated by adding neomycin (1.5 mg/ml) to the medium. Transfectants designated 10vRA, 15vRA and 20vRA were used in some experiments.

(iii) Binding of Anti-CD44 mAbs to Namalwa Transfectants

Flow cytometry : Namalwa transfectants were incubated with anti-CD44s mAb (Serotec) or with anti-CD44v6 mAb (Bender Med System) for 45 min at 4° C., washed three times with PBS and reincubated with anti-mouse Fab′-FITC (Jackson) for 30 min at 4° C. Then the cells were washed and analyzed by flow cytometry for their ability to bind the antibodies.

Western Blot: Cell lysates were loaded on SDS-PAGE. Following electrophoresis, the separated proteins were transferred to a nitrocellulose membrane, immunobloted with anti-CD44 mAb (Hermes-3) and exposed to X-ray film.

(iv) Binding of FGF-2 to Namalwa Transfectants

Flow cytometry : A quantity of 20 ng biotinilated FGF-2 were incubated with 10⁶ Namalwa transfectants for 45 min at 4° C., washed three times with PBS, incubated with streptavidin—PE (Jackson) for 30 min at 4° C., and analyzed for their ability to bind FGF-2 by flow cytometry. For blocking the binding of the FGF-2, the cells were preincubated with 20 μg/ml heparin or chondroitin sulfate A and C (Sigma) for 45 min at 4° C., washed three times with PBS, incubated with biotinilated FGF-2, as indicated above, washed, and then incubated with streptavidin-conjugated phycoerythrin (streptavidin-PE) (Jackson) for 30 min at 4° C. and analyzed by flow cytometry for their ability to bind FGF-2. For enzymatic treatment, the cells were incubated with 10 mμ/ml heparinase I or with 100 mμ/ml chondroitinase ABC (Sigma) for 2 h at 37° C. Then, the cells were washed three time and incubated with 20 ng biotinilated FGF-2 for 45 min at 4° C., washed again three times with PBS, incubated with streptavidin-PE (Jackson) for 30 min at 4° C. and analyzed for their ability to bind FGF-2 by flow cytometry.

Western Blot: Namalwa transfectants were incubated with FGF-2 (200 ng/10⁶ cells) for 1 hour at 4° C. The cells were washed once with 0.2 M NaCl and twice with PBS, lyzed with NP-40 buffer, and the cell lysate was loaded on 12% SDS-PAGE. Following electrophoresis the separated proteins were transferred to a nitrocellulose membrane, immunobloted with anti-FGF-2 mAb (Serotec) and exposed to X-ray film.

(v) FGFR-1 Binding Assay

For this assay, medium conditioned by cells expressing soluble FGF receptor 1-alkaline phosphatase fusion protein (FGFR1-AP), prepared as described (Aviezer et al., 1994a), was used. Thus, 200 ng bFGF and conditioned medium containing FGFR1-AP were mixed with 10⁶ Namalwa transfectants, incubated for 3 h at 4° C. and washed three times with PBS. Then, p-nitrophenyl phosphate (NPP) substrate (Sigma) was added to the cells for 3 h at 37° C. allowing the color to be developed prior to spectrophotometry reading at 405 nm. As a control, 10⁶ Namalwa transfectants were incubated with 20 ng of biotinilated FGF-2 for 45 min at 4° C., washed three times with PBS, and incubated with streptavidin-AP (Jackson) for 30 min at 4° C. and rewashed three times with PBS. Then pNPP substrate was added to the cells for 1 h at 37° C. and fluorescence was detected as above at 405 nm.

Synoviocytes from rheumatoid arthritis and osteoarthritis patients were washed in PBS and incubated with FGFR1-AP for 4 hours, washed three times with PBS. Then pNPP substrate was added to the cells for 3 h at 37° C. allowing the color to be developed, prior to analysis in spectrophotometer reader at 405 nm. For blocking assay with antibodies, anti-CD44v3 mAb (R&D) or anti-CD44s mAb (Serotec) or Isotype matched control immunoglobulin (IGg 2b; Serotec) were added to the cells before addition of FGFR1-AP, and then exposed to the detection system, as indicated above.

(vi) BaF32 Cell Proliferation (Transactivation) Assay

Namalwa transfectants were fixed with paraformaldehyde (1% w/v in PBS, 2 h, 4° C.), washed three times with cold PBS and suspended in RPMI 1640 containing 0.5% FCS. Fixed cells (50 μl, 3×10⁶/ml) were then mixed, in 96-well microtiter plates, with an equal volume of BaF32 cells (3×10⁵/ml) and recombinant FGF-2 (10 nM, final concentration) and incubated for 72 h at 37° C. At the end of this period, 20 μl of MTS (Promega) was added to each well and the plate developed at 37° C. for 1 h prior to spectrophotometry plate-reader at 490 nm.

### Example 1

### Rheumatoid Arthritis Specific-CD44 Variant—CD44vRA

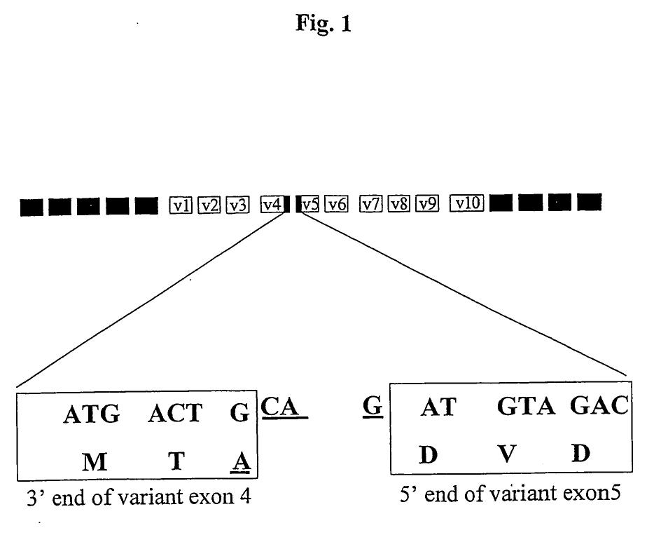

In searching for disease-specific CD44 isoforms in RA patients, RT-PCR revealed CD44 variant transcripts, mostly CD44v3-v10, in 44 of 47 RA patients subjected to this test. The CD44v3-v10 was also identified in normal keratinocytes. When the CD44v3-v10 isoform of RA synoviocytes was sequenced, we discovered that it included an extra trinucleotide sequence (CAG) that was illegitimately transcribed from the end of intron bridging exon v4 to exon v5, allowing to encode alanine, without interfering with the entire reading frame (FIG. 1). A transcript with identical sequence change, designated CD44vRA (CD44 variant of RA patients), was detected in 20 of 26 RA patients (not shown).

The entire hCD44v3-10 cDNA was cloned from human keratinocyte total RNA by reverse transcriptase-polymerase chain reaction (RT-PCR) amplification, using two primers assigned from the published CD44 sequence, including the Xbal recognition site, as described in WO 00/75312. The PCR product was digested with Xbal enzyme and ligated into a pcDNA3.1 Neovector (Invitrogen).

Using the same protocol the entire hCD44vRA cDNA was cloned from cellular total RNA extracted from synovial cells of rheumatoid arthritis patients and was subjected to RT-PCR. The pcDNA3.1 neovector alone served as control.

### Example 2

### CD44v3-v10 Variants Bind to the Same Extent FGF-2

Namalwa cells (derived from patients with Burkitt's lymphoma), lacking CD44, were transfected with: (1) standard CD44 cDNA isolated from Hela cell line (designated Namalwa CD44s), (2) CD44v3-v10 cDNA isolated from keratinocytes (Namalwa-CD44v3-v10 or Namalwa-CD44v), (3) CD44v3-v10 cDNA containing the extra CAG isolated from RA synoviocytes (Namalwa-CD44vRA), or (4) with empty vector (Namalwa-Neo).

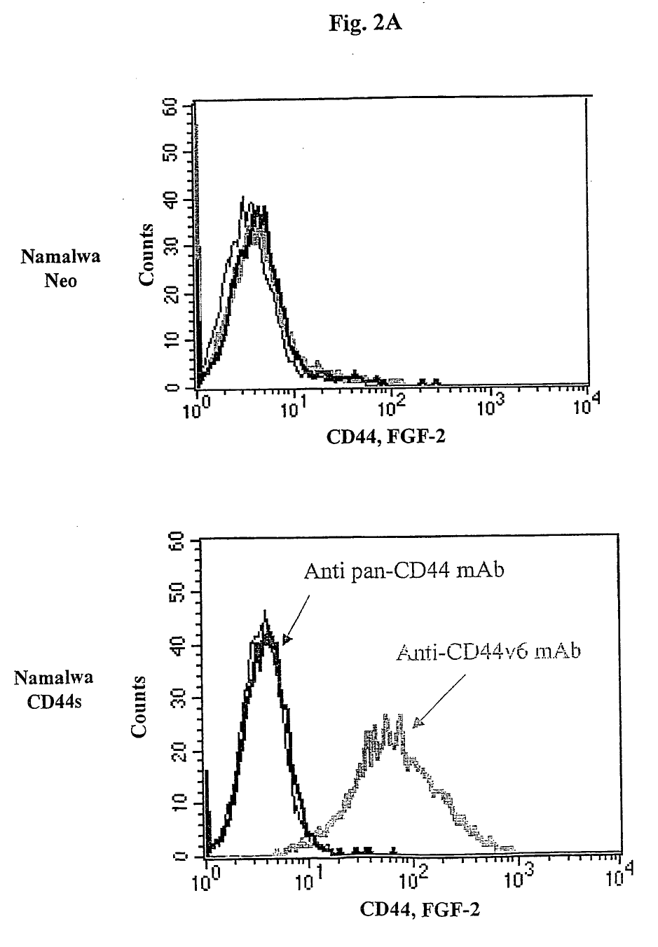

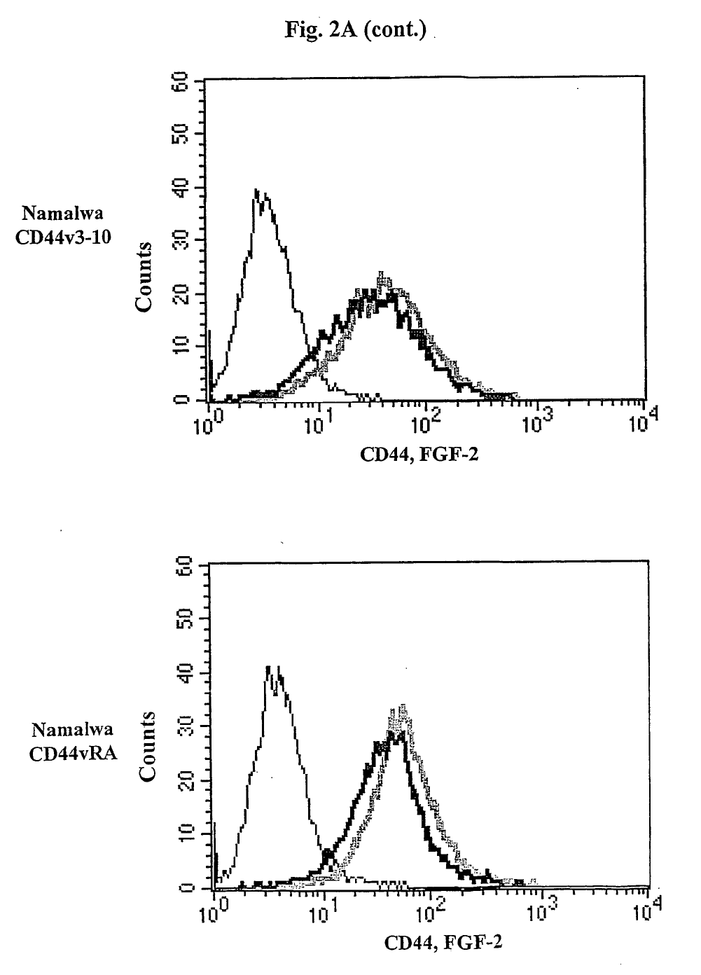

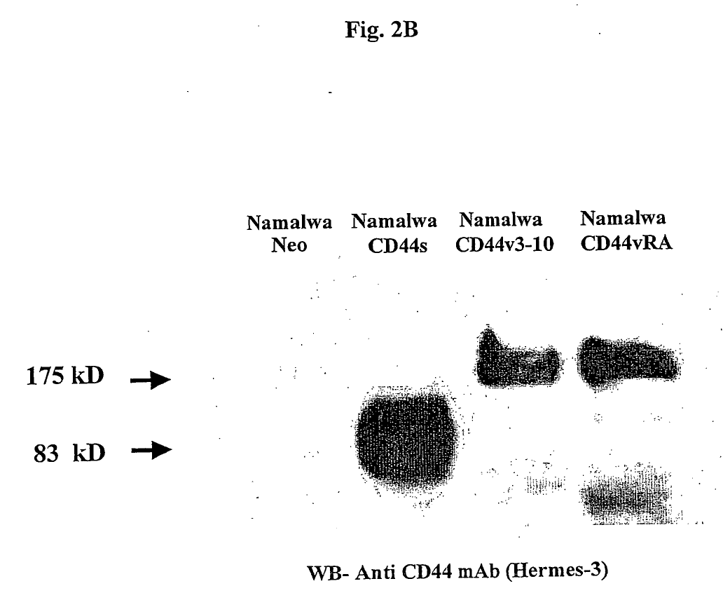

As shown in FIG. 2A, P1 anti-constant CD44 mAb almost equally stained Namalwa-CD44s, Namalwa-v3-v10 and Namalwa-CD44v RA cells, but not Namalwa-Neo cells. On the other hand, anti-CD44v6 mAb similarly stained Namalwa-CD44v3-v10 and Namalwa-CD44vRA cells, but did not stain Namalwa-CD44s or Namalwa-Neo cells. These results were confirmed by Western Blot analysis with Hermes-3 anti-CD44 mAb, showing that extract of Namalwa-CD44s includes a CD44s only, and extracts of Namalwa-CD44v3-v10 as well as Namalwa-CD44vRA contain a CD44 variant only. Note that Namalwa-CD44v3 -v10 and Namalwa-CD44vRA present similar quantity of CD44 protein, while Namalwa-CD44s cells contain a higher level.

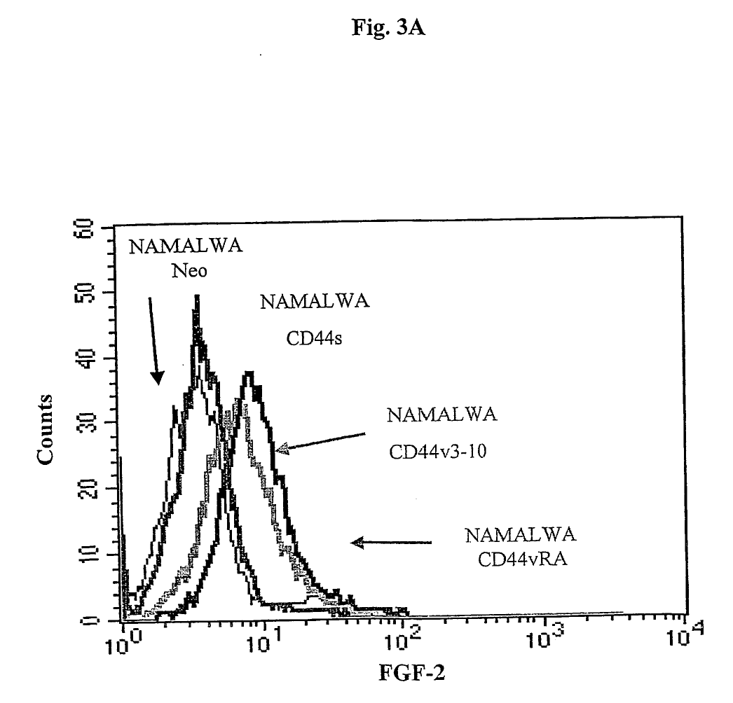

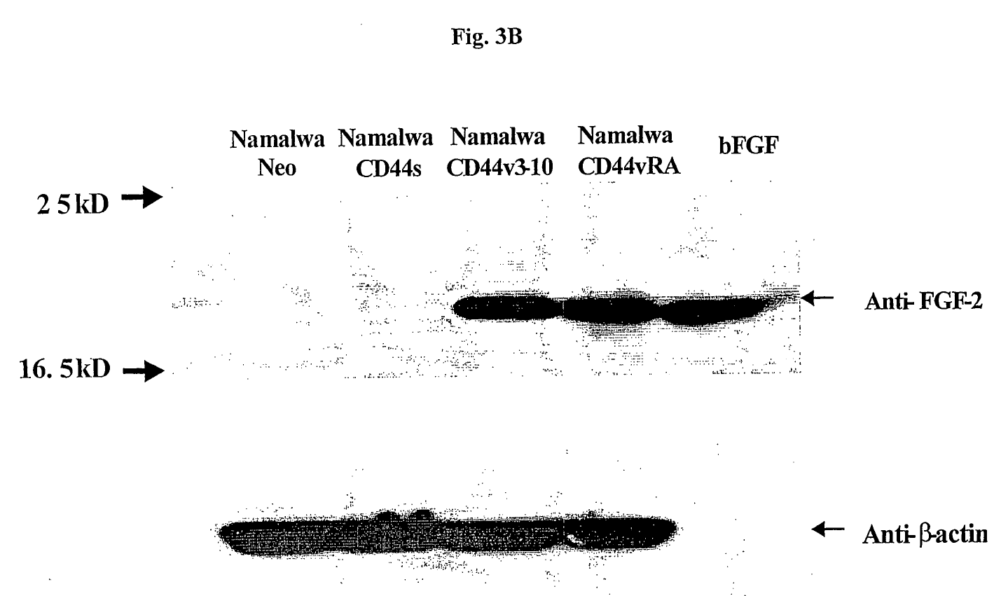

Heparin-binding growth factors (e.g., FGF-2) or chemokines can be bound to v3 heparan sulfate of v3-containing CD44 variants (but not to other variant exons) and then presented autocrinically or paracrinically to the corresponding receptors. Flow cytometry analysis reveals that fluorescein-labeled FGF-2 displays close to equal enhanced binding to Namalwa-CD44v3-v10 cells or to Namalwa-CD44vRA cells, when compared to Namalwa-CD44s or Namalwa-Neo cells, which show less efficient binding (FIG. 3A). The similar binding of FGF-2 to Namalwa CD44v3-v10 or Namalwa- CD44vRA was confirmed by Western Blot analysis with anti-FGF-2 antibody, using cell extracts from Namalwa transfectants expressing the corresponding CD44 variants (FIG. 3B).

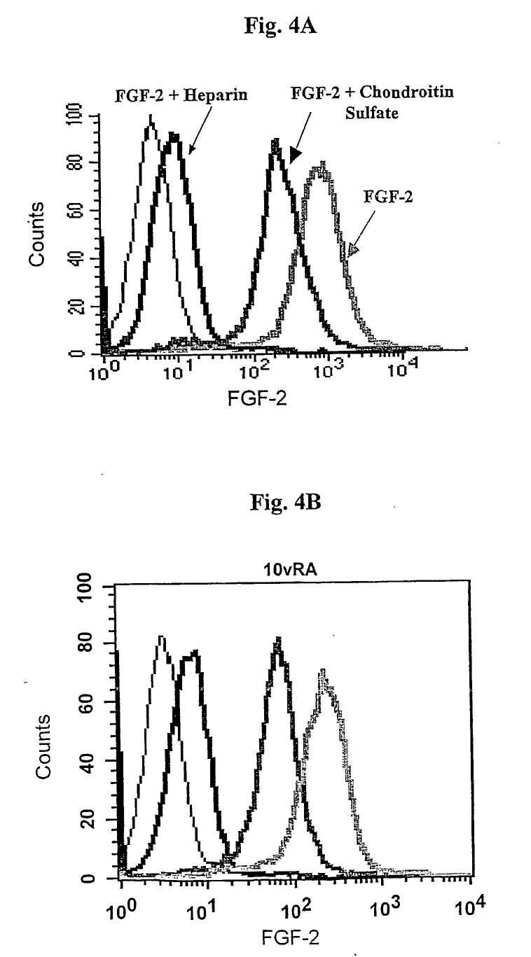

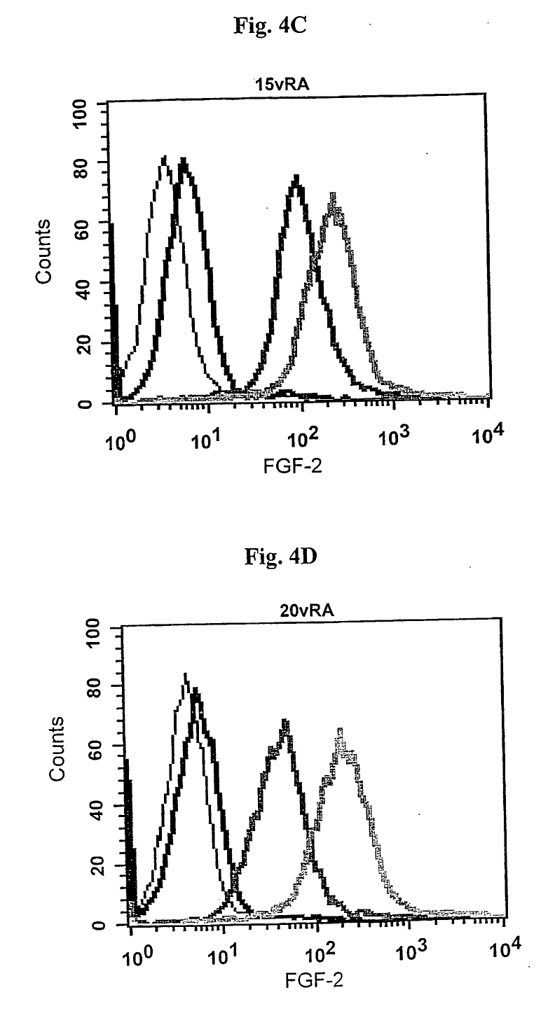

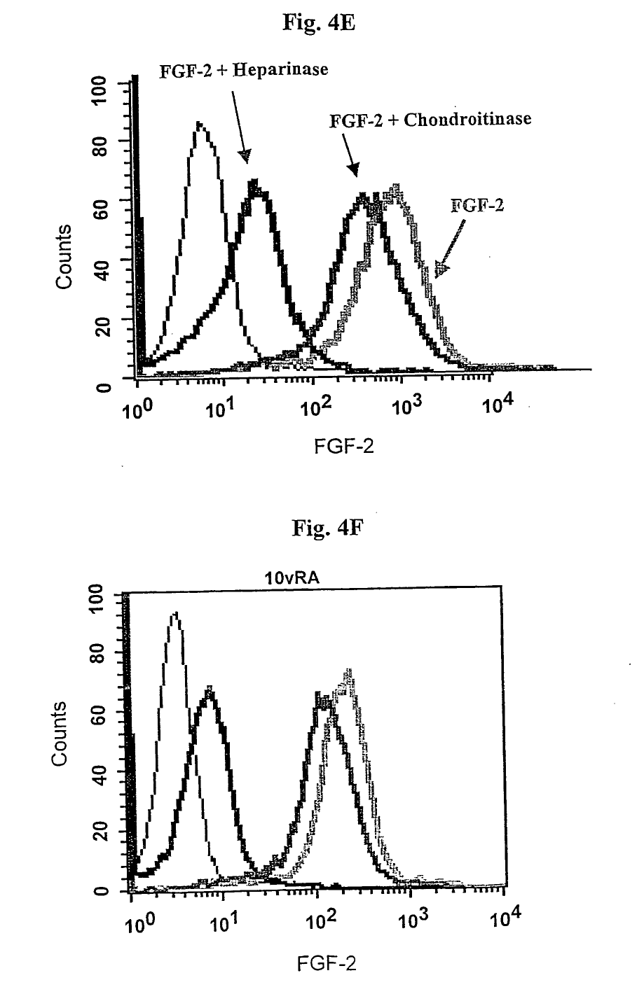

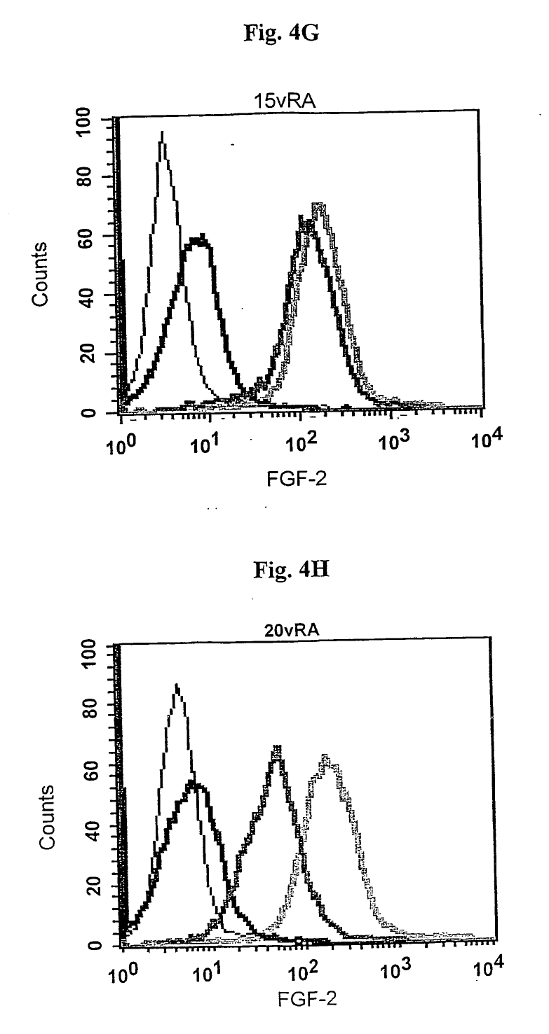

The binding of fluorescein-labeled FGF-2 to Namalwa-CD44vRA (FIG. 4A) and three of its clones—10vRA, 15vRA and 20vRA (FIGS. 4B-4D, respectively) was blocked by an excess of soluble heparin, but much less when soluble chondroitin sulfate was used for blocking (FIG. 4A), indicating that heparan sulfate mediates the interaction between FGF-2 and Namalwa-CD44vRA. Similarly, the binding of fluorescein-labeled FGF-2 to Namalwa-CD44vRA (FIG. 4E) and three of its clones—10vRA, 15vRA and 20vRA (FIGS. 4F-4H, respectively) was inhibited by pretreatment of the cells with heparinase, but not with chondroitinase ABC (FIG. 4E), confirming the involvement of heparin in FGF-2 binding to the cell surface CD44 variant.

### Example 3

### FGF-2 Bound To Cell Surface CD44vRA Induced Enhanced Stimulation Of Its Receptor

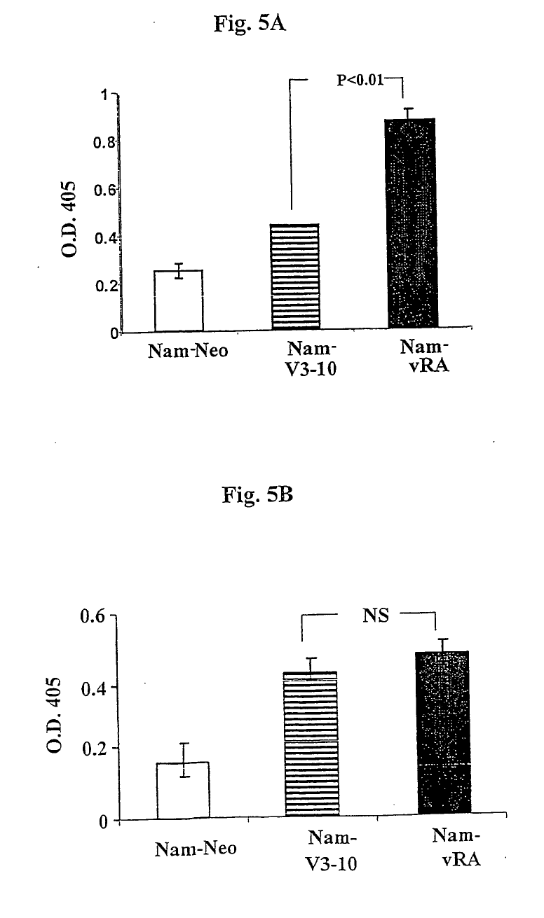

In contrast to soluble alkaline phosphatase-labeled FGF-2 (FIG. 5B), soluble alkaline phosphatase-labeled FGF receptor 1 showed better binding to Namalwa-CD44vRA preincubated with FGF-2, than to similarly treated Namalwa-CD44s or Namalwa-CD44v3-v10 (FIG. 5A). This finding suggests that the orientation (and not the concentration) of heparin-bound FGF-2 on cell surface CD44vRA allows enhanced interaction with its receptor.

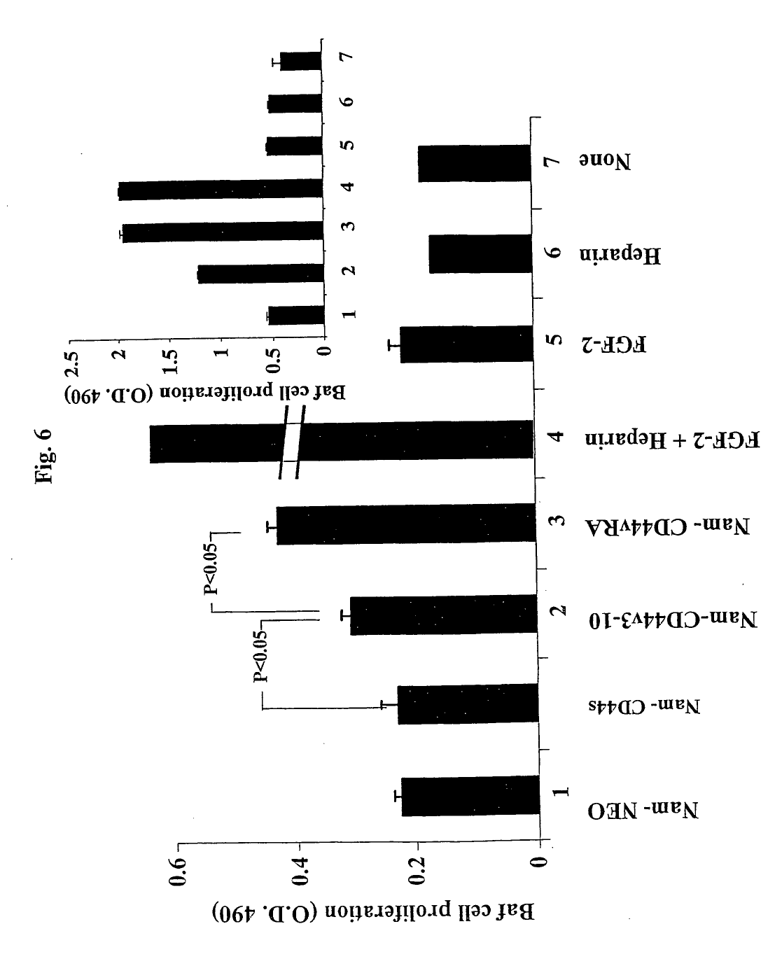

Indeed, addition of FGF-2 to fixed Namalwa-CD44vRA cells allows its efficient presentation to BaF-32 cells expressing the FGF receptor 1. This well-oriented FGF-2 presentation resulted in enhanced cell proliferation of BaF-32 cells, similarly to that of BaF-32 cells incubated with FGF-2 and heparin. On the other hand, BaF-32 cells proliferated less intensively when incubated in the presence of FGF-2 with fixed Namalwa-CD44s or Namalwa-CD44v3-v10 cells. In contrast, BaF-32 cells incubated with FGF-2 alone or heparin alone showed a background level of cell proliferation, similarly to the proliferation rate of BaF-32 cells co-cultured, in the presence of F-2GF, with Namalwa-Neo cells (FIG. 6 and inset).

### Example 4

### Enhanced CD44-associated Binding of FGF Receptor-1 to Synovial Fluid Cells of RA Patients

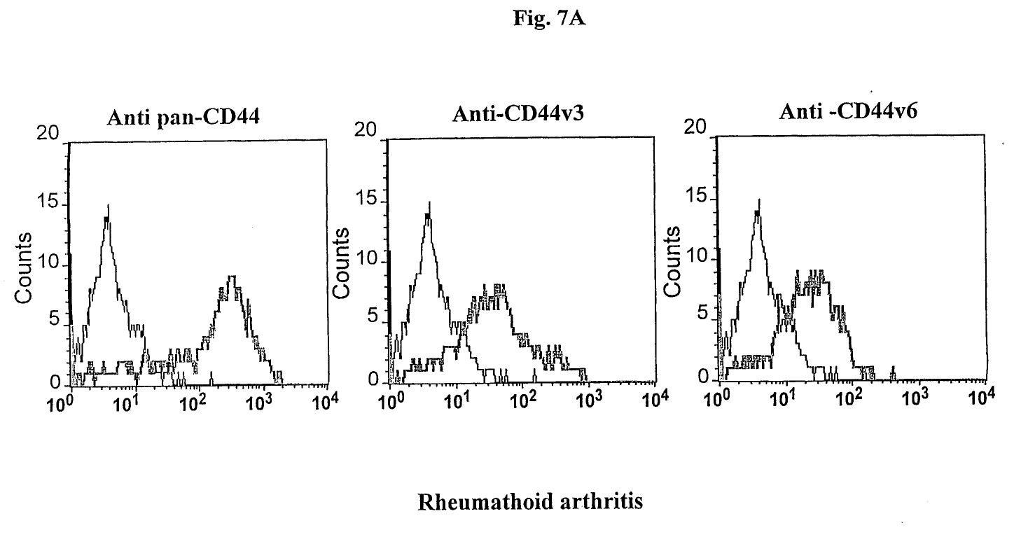

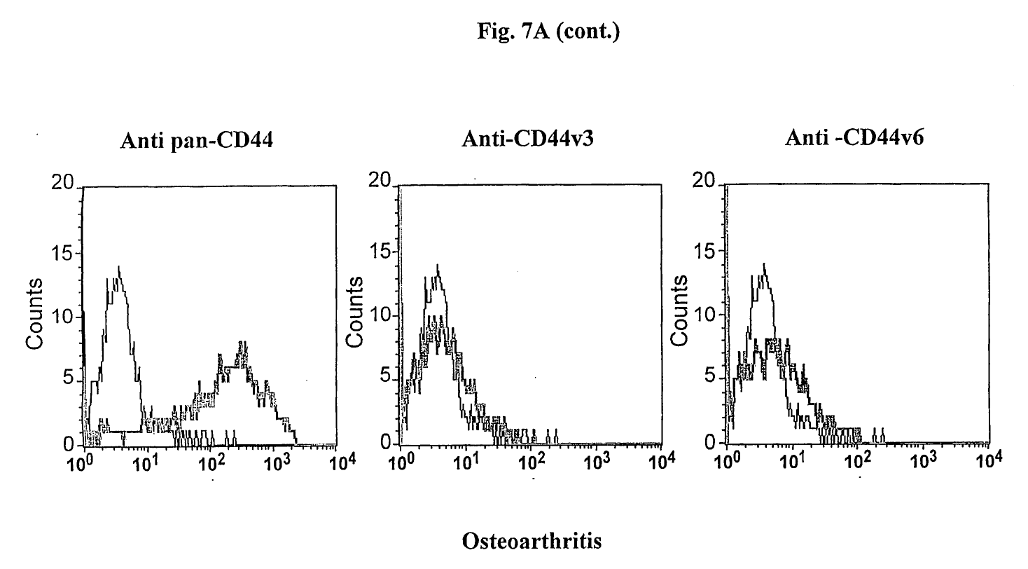

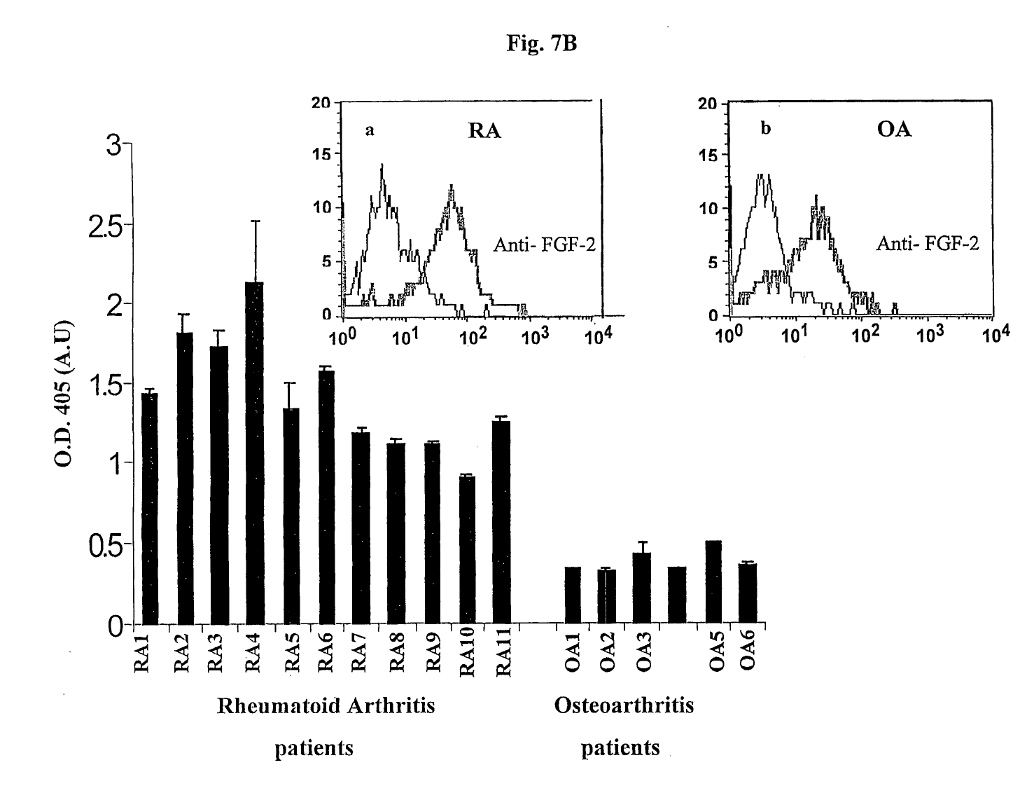

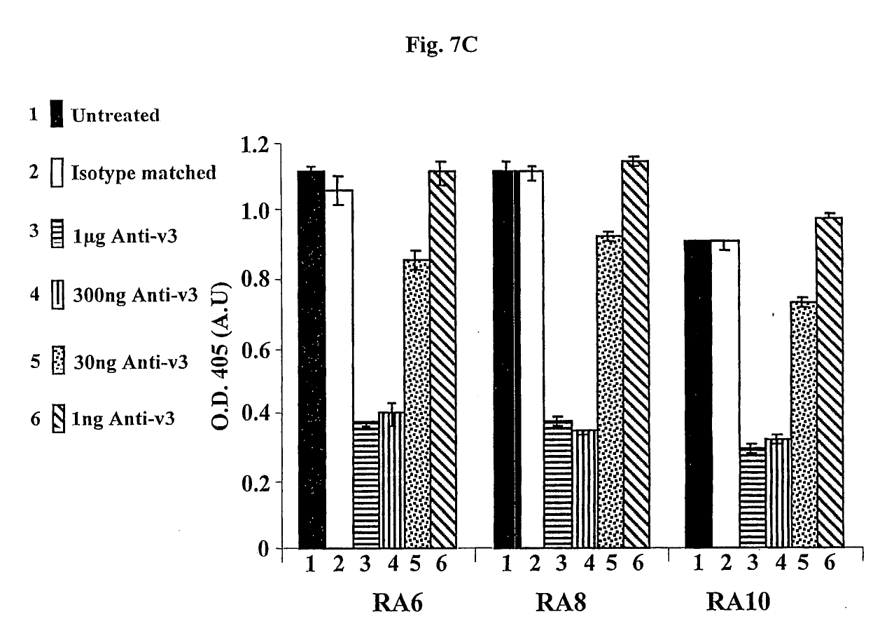

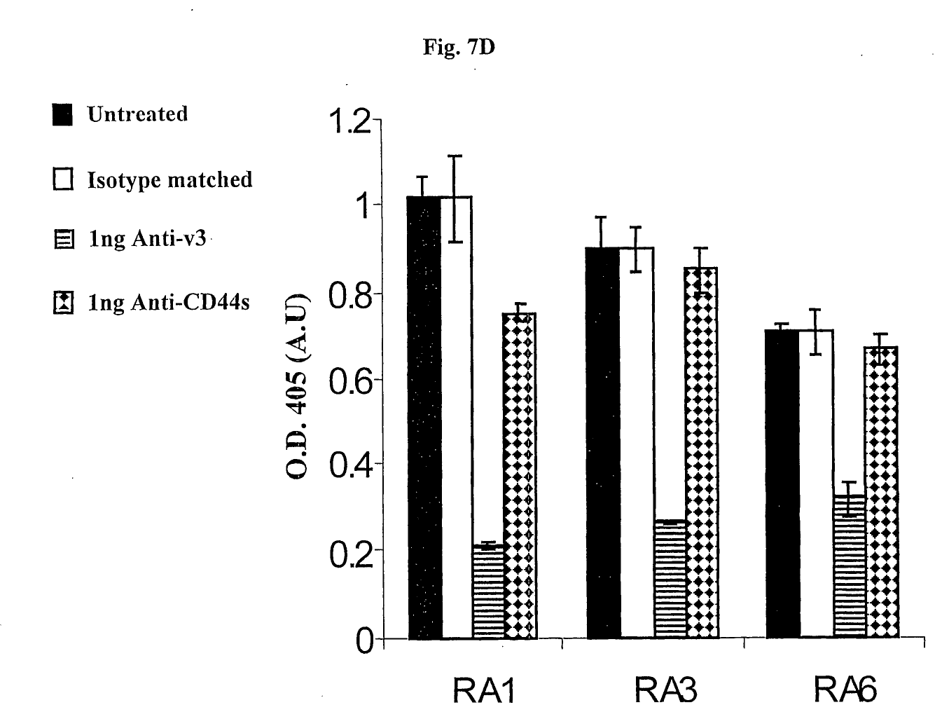

Binding of FGF-2 and FGF receptor-1 to Namalwa cells expressing CD44 isoforms is an artificial model of receptor-ligand interaction. Therefore, it was important to confirm the findings in authentic cells taken from the joints of patients with joint diseases. Flow cytometry analysis shows that although joint cells of RA and OA patients almost equally express pan-CD44 receptor, only RA joint cells express CD44v3 and CD44v6 epitopes, as indicated by immunostaining with the corresponding mAbs (FIG. 7A). Furthermore, immunostaining with anti-FGF-2 antibody to detect inclusion of FGF-2 on the cell surface of joint cells reveals that RA and OA joint cells equally contain the same level of FGF-2 (FIG. 7B, insets). The histograms depicted in insets a and b of FIG. 7B represent 11 RA patients (a) and 6 OA (b) patients, respectively. However, RA joint cells (derived from 11 different patients) bind soluble FGF receptor-1 more impressively than OA joint cells (derived from 6 different patients) regardless as to whether FGF-2 was added (not shown) or not added (FIG. 7B) to the cells. These results indicate that joint FGF-2 is endogenously bound to the joint cells (as shown in FIG. 7B insets) and they further suggest that the cell surface orientation of FGF-2 binding is more important for the interaction with the corresponding receptor than the cell surface concentration of this growth factor. The binding of soluble FGF receptor-1 to synovial fluid cells of RA patients was inhibited, in dose-dependent manner, with anti-CD44v3 mAb (FIG. 7C) but not with anti-pan CD44mAb (FIG. 7D), indicating that the FGF receptor-1 binding is CD44-associated.

### Example 5

### Preparation of Soluble hCD44v3-10, hCD44vRA and hCD44s Plasmids

The soluble CD44v3-10 cDNA (nucleotide sequence 1-1824 from the published sequence of CD44 by Screaton et al, 1992) was cloned from total RNA of primary human keratinocyte by RT-PCR amplification, using two primers assigned from said published CD44 sequence:

| | |
|---|---|
| Ex1s: | |
| TATCTAGAGCCGCCACCATGGACAAGTTTTGGTGG | (SEQ ID NO:3) |
| Ex16/17 as: | |
| TATCTAGAGCCATTCTGGAATTTGGGGTGT | (SEQ ID NO:4) |

Both primers contained the Xbal recognition site. Using the same protocol, the soluble CD44vRA cDNA (SEQU ID NO:1) and soluble CD44s cDNA were cloned from synovial cells of a rheumatoid arthritis patient.

The PCR products were digested with Xbal enzyme and pCXFc zeovector was digested with Nhel restriction enzyme. After digestion, the PCR products were ligated into the pCXFc zeovector containing the Fc region of the gamma globulin heavy chain.

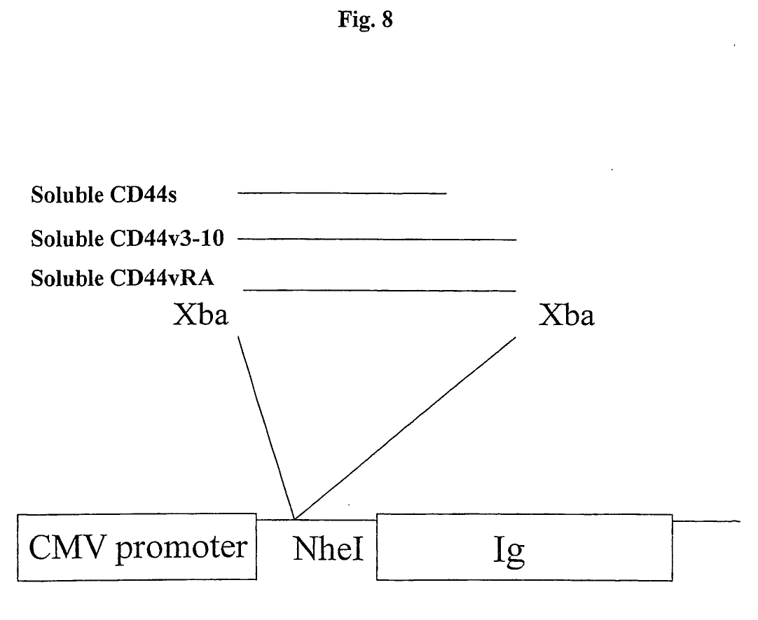

A schematic description of the pCXFc-CD44 zeovector is shown in FIG. 8.

### Example 6

### Transient Transfection of the CD44 Plasmids Into 293T Cells

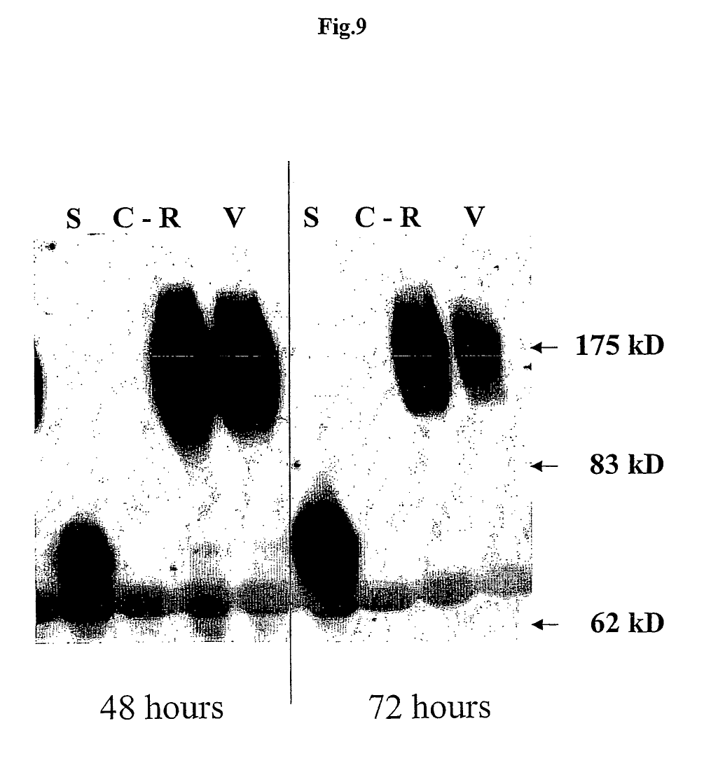

For transient transfection into 293T cells, 3 μg of each plasmid of Example 5 was incubated for 20 min with 12 μl of FuGene (Roche). The mixture was added into 15-cm cell plates containing 70% confluent of 293T cells. The soluble fusion CD44-Fc proteoglycans obtained were purified on Protein G column, supernatants were collected after 48 h and 72 h, added to a SDS-PAGE gel and then transferred to nitrocellulose membrane for immunobloting with anti-CD44 mAbs (Hermes-3). The results are shown in FIG. 9. The SDS-PAGE analysis reveals that 293T cells transfected with soluble CD44s-Ig DNA (S, first lane) release soluble CD44s protein (70 kDa), whereas 293T cells transfected with soluble CD44v3-v10 cDNA (V, lane 4) or soluble CD44vRA cDNA (V, lane 3) release the corresponding variant proteins (175 kDa). Control (C. lane 2)—non-transfected cells. Molecular weight markers are indicated.

### DISCUSSION

For efficient autocrinic or paracrinic presentation to the relevant neighboring receptors, FGF-2, like other heparin-binding growth factors, must be assembled on cell surface heparan sulfate proteoglycan. The proteoglycanic nature of cell surface CD44 has been well established. CD44 includes seven potential consensus single serine-glycine (SG) or double SGSG assembly sites for GAGs, e.g heparan sulfate, chondroitin sulfate, keratin sulfate, attachment. However, it was found that the assembly of HS, a member of the GAG family, is determined by eight amino acids located downstream to the SGSG motif. As exon v3 of the CD44 proteoglycan is the only one containing this sequence in the context with SG or SGSG motif, heparan sulfate assembly is merely confined to this exon. The sequestering of HS-binding growth factors, including FGF-2, FGF-4 and FGF-8, on the v3 exon of CD44 proteoglycan has been well documented. We have shown herein in the specification (FIG. 7) that anti-CD44v3 mAb inhibited the binding of soluble FGF receptor 1 to synovial fluid cells of RA patients. Accumulated findings show unequivocally that the growth factor linker HS is attached to the v3 exon, including v3 exon expressed on Namalwa cells. It is also shown (FIG. 7) that inflamed synovial membrane macrophages express v3-containing CD44 HS proteoglycan.

### REFERENCES

Aviezer, D. et al., 1994. Differential structural requirements of heparin and heparan sulfate proteoglycans that promote binding of basic fibroblast growth factor to its receptor. J. Biol. Chem. 269(1): 114-121.

Brocke, S., et al. 1999. Antibodies to CD44 and integrin α, but not L-selectin, prevent central nervous system inflammation and experimental encephalomyelitis by blocking secondary leukocyte recruitment. Proc. Natl. Acad. Sci. USA 96: 6896.

Nader, H. B., Dietrich, C. P., Buonassisi, V., and Colbrun, P. (1987) Proc. Natl. Acad. Sci. USA 84, 3565-3569.

Naor, D., Vogt Sionov, R., and Ish-Shalom, D. 1997. CD44: structure, function and association with the malignant process. Adv. Cancer Res. 71: 241.

Nedvetzki, S., Walmsley, M., Alpert, E., Williams, R. O., Feldmann, M., and Naor, D. 1999. CD44 involvement in experimental collagen-induced arthritis (CIA). J. Autoimmunity 13: 39.

Rapraeger, A. C., Krufka, A, and Olwin, B. 1991. Requirement of heparan sulfate for bFGF-mediated fibroblast growth and myoblast differentiation. Science 252: 1705-1708.

Screaton, G. R., Bell, M. V., Jackson, D. G., Cornelis, F. B., Gerth, U., and Bell, J. I. 1992. Genomic structure of DNA encoding the lymphocyte homing receptor CD44 reveals at least 12 alternatively spliced exons. Proc. Natl. Acad. Sci. USA 89:12160.

Screaton, G. R., Bell, M. V., Bell, J. I., and Jackson, D. G. 1993. The identification of a new alternative exon with highly restricted tissue expression in transcripts encoding the mouse Pgp-1 (CD44) homing receptor. Comparison of all 10 variable exons between mouse, human and rats. J. Biol. Chem. 268:12235.

Stickeler, E., et al. 2001. Modulation of soluble CD44 concentrations by hormone and anti-hormone treatment in gynecological tumor cell lines. Oncology Reports 3: 1381-1386.

Weiss, L., Slavin, S., Reich, S., Cohen, P., Shuster, S., Stem, R., Kaganovsky, E., Okon, E., Rubinstein, A. M., and Naor, D. 2000. Induction of resistance to diabetes in non-obese diabetic mice by targeting CD44 with a specific monoclonal antibody. Proc. Natl. Acad. Sci. USA, 97:285.

Yayon et al., 1991. Cell-surface, heparin-like molecules are required for binding of basic fibroblast growth factor to its high affinity receptor. Cell 64: 841-848.

1-7: (canceled)

8: An agent capable of modulating the activity of a heparin-binding growth factor (HBGF) by enhancing or inhibiting high affinity binding of said HBGF to its receptor, said agent being selected from the group consisting of:
(i) the soluble CD44vRA variant expressed in synovial cells of rheumatoid arthritis (RA) patients (herein CD44vRA), carrying at least one chain of a heparan sulfate;
(ii) a recombinant chimeric fusion protein comprising the amino acid sequence of soluble CD44vRA variant fused to a tag suitable for proteoglycan purification, said fusion molecule being post-translationally glycosylated to carry at least one chain of a heparan sulfate; and
(iii) a sugar molecule being a heparan sulfate derived from a CD44 isoform, or a fragment thereof.

9: A recombinant chimeric fusion protein of claim 8 wherein the amino acid sequence of the soluble CD44vRA (SEQ ID NO:2) is fused to a tag selected from the group consisting of the Fc region of the gamma globulin heavy chain, glutathione S-transferase (GST) or polyHis.

10: The recombinant chimeric fusion protein of claim 9 wherein soluble CD44vRA is fused to the Fc region of the gamma globulin heavy chain (CD44vRA-Fc).

11: A heparan sulfate of claim 8 derived from a CD44 isoform carrying at least one chain of a heparan sulfate, or a fragment thereof.

12: A heparan sulfate of claim 11, derived from a CD44 isoform selected from the group consisting of CD44s, CD44v3-v10, and CD44vRA.

13: A heparan sulfate of claim 12, wherein said CD44 variant is CD44vRA.

14: An agent according to claim 8, wherein said at least one heparan sulfate chain has at least one highly sulfated domain.

15: An agent according to claim 8, wherein said at least one heparan sulfate chain contains at least 2 monosaccharide residues.

16: An agent according to claim 26, wherein said heparan sulfate chain contains 10-16 monosaccharide residues.

17: An inhibitor of a heparan sulfate agent as defined in claim 8(iii), being selected from the group consisting of an antibody, a peptide, an oligosaccharide or a polysaccharide mimetic.

18: A method for diagnosis of rheumatoid arthritis in an individual comprising:
(i) obtaining a sample from the joints of the individual;
(ii) contacting said sample with a FGFR conjugated to a detection system;
(iii) detecting the presence of CD44vRA expressed by the joint cells in the sample through binding of an endogenous or exogenous FGF and the conjugated receptor by adding a substrate for the detection system, whereby development of color indicates the presence of CD44vRA expressed by the joint cells and a high probability of rheumatoid arthritis.

19-25: (canceled)

26: An agent according to claim 8, wherein said at least one heparan sulfate chain contains at least 6 monosaccharide residues.

27: A method for modulation of the activity of a heparin-binding growth factor (HBGF) by enhancing or inhibiting high affinity binding of said HBGF to its receptor, which comprises administering to an individual in need an agent selected from the group consisting of:
(i) a soluble CD44 isoform carrying at least one chain of a heparan sulfate;
(ii) a recombinant chimeric fusion protein comprising the amino acid sequence of a soluble CD44 isoform fused to a tag suitable for proteoglycan purification, said fusion molecule being post-translationally glycosylated to carry at least one chain of a heparan sulfate; and
(iii) a sugar molecule being a heparan sulfate derived from a CD44 isoform, or a fragment thereof,
in an amount effective to enhance or inhibit high affinity binding of said HBGF to its receptor in said individual.

28: A method according to claim 27, wherein said HBGF is a growth factor selected from the group consisting of a member of the fibroblast growth factor (FGF), colony-stimulating factor (CSF), transforming growth factor beta (TGF-β), interleukin (IL), or bone morphogenetic protein (BMP) families, heparin-binding epidermal-like growth factor (HB-EGF), insulin-like growth factor (IGF), vascular endothelial growth factor (VEGF), macrophage inflammatory protein-1β (MIP-1β), regulated on activation, normally T cell expressed and secreted (RANTES), and hepatocyte growth factor (HGF).

29: A method according to claim 27, for modulating heparin-dependent growth factor activity relevant for promoting tissue-specific cell proliferation, migration and differentiation.

30: A method according to claim 29, for induction of angiogenesis and blood vessel formation, bone fracture healing, enhancement of wound healing, treatment of ischemic heart diseases and peripheral vascular diseases, neuronal regeneration, and promotion of tissue regeneration.

31: A method according to claim 30 wherein the promotion of tissue regeneration consists in promotion of liver regeneration, or promotion of tissue regeneration after transplantation of myocytes into heart tissues or of dopaminergic/neuronal cells into brain tissue.

32: A method according to claim 30, comprising the administration of said agent together with a compound selected from the group consisting of a FGF, a CSF, a TGF-β, an IL, VEGF, MIP-1β, BMP, IGF, HB-EGF, RANTES and HGF.

33: A method according to claim 27 for prevention and treatment of infectious and other inflammatory diseases, autoimmune diseases and CD44-dependent cancer.

34: A method for the treatment of rheumatoid arthritis comprising administering to a patient in need the soluble CD44vRA variant expressed in synovial cells of rheumatoid arthritis (RA) patients (herein CD44vRA), carrying at least one chain of a heparan sulfate, in an amount effective to treat rheumatoid arthritis in said patient.
